(19) United States
(12) Patent Application Publication
(10) Pub. No.: US 2006/0148826 A1
(43) Pub. Date: Jul. 6, 2006

(54) Stable aqueous solutions of risperidone and methods for their preparation

(76) Inventors: Ashish Gogia, New Delhi (IN); Sunilendu Bhushan Roy, Maharashtra (IN); Rajiv Malik, Vienna (AT)

Correspondence Address: RANBAXY INC., 600 COLLEGE ROAD EAST, SUITE 2100, PRINCETON, NJ, 08540, US

(21) Appl. No.: 10/525,166

(22) PCT Filed: Aug. 25, 2003
(86) PCT No.: PCT/IB03/03529

§ 371 Date: Feb. 27, 2006

(30) Foreign Application Priority Data
Aug. 23, 2002 (IN) 859/DEL/2002

(51) Int. Cl. A61K 31/519; A61K 31/7012; A61K 31/70

(52) U.S. Cl. 514/259.41; 514/23; 514/53

(57) ABSTRACT

The technical field of the present invention relates to stable aqueous solution of risperidone for oral administration; and process for preparation thereof.

### FIELD OF THE INVENTION

The technical field of the present invention relates to stable aqueous solution of risperidone for oral administration; and process for preparation thereof.

### BACKGROUND OF THE INVENTION

Risperidone, chemically 3-[2-[4-(6-fluoro-1,2-benzisoxazol-3yl)-1-piperidinyl]ethyl]-6,7,8,9-tetrahydro-2-methyl-4H-pyrido[1,2-a]pyrimidin-4-one belongs to a new chemical class of antipsychotic agents. It is indicated for the management of the manifestations of psychotic disorders. Oral solutions of risperidone are commercially marketed by Janssen Pharma under the trade name Risperdal®.

U.S. Pat. No. 4,804,663 discloses 3-piperidinyl-1,2-benziosoxazolles and their pharmaceutically acceptable acid addition salts having useful antipsychotic activity. It also exemplifies an oral solution of the above compounds with preservatives, tartaric acid, sodium-saccharin, flavors, and the polyhydric alcohols such as sorbitol and glycerol.

However, as disclosed in U.S. Pat. No. 5,453,425, comparable solutions in which the benzisoxazole derivative was risperidone exhibited an unsatisfactory physicochemical stability. The instability was found to be caused due to sorbitol, which accelerated the decomposition of risperidone upon storage at elevated temperatures. A similar observation was made with maltitol, suggesting that risperidone is incompatible with polyhydric alcohols.

The above incompatibilities have been addressed in the prior art by avoiding the use of polyhydric alcohols in pharmaceutical compositions of risperidone. For example, U.S. Pat. Nos. 5,453,425 and 5,616,587 disclose aqueous solutions of risperidone essentially free of polyhydric alcohols, such as mannitol, fructose, sucrose, maltose and the like.

Polyhydric alcohols have several advantages and form a class of one of the most widely used sweeteners or bitter taste-masking agents in oral liquid dosage forms. Hence, one would generally desire to have the option of using polyhydric alcohols as sweeteners in forming stable liquid dosage forms of risperidone.

### SUMMARY OF THE INVENTION

In one general aspect there is provided an aqueous solution of risperidone. The aqueous solution includes water; a therapeutically effective amount of risperidone or a pharmaceutically acceptable free risperidone base or acid addition salt of risperidone; one or more polyhydric alcohols; and one or more buffering agents configured to maintain the pH in the range of about 2 to about 6.

Embodiments of the solution may include one or more of the following features. For example, the addition salt may be selected from one or more of salts of risperidone with inorganic acids comprising hydrochloric, hydrobromic, sulfuric, nitric, and phosphoric acids; or organic acids comprising acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, and pamoic acids. The one or more polyhydric alcohols may be one or more of monosaccharides, disaccharides and sugars. The monosaccharide may be one or both of glucose (dextrose) and fructose (levulose). The disaccharide may be one or more of sucrose, lactose, maltose and cellobiose. The disaccharide may be sucrose. The sugars may be one or more of ribose, glycerine, sorbitol, xylitol, maltitol, erythritol, inositol, lactitol monohydrate, propylene glycol, galactose, mannose, xylose, rhamnose, glutaraldehyde, invert sugars, mannitol, polyethylene glycol and glycerol. The sugar may be sorbitol.

The aqueous solution may further include an antioxidant. The antioxidant may be one or more of antioxidants, reducing agents and antioxidant synergist. The antioxidants may be one or more of acetylcysteine, alpha tocopherol acetate, d-alpha tocopherol, dl-alpha tocopherol, ascorbyl palmitate, butylated hydroxyanisole (BHA), butylated hydroxytoluene (BHT), cysteine, cysteine hydrochloride and propyl gallate. The reducing agent may be one or more of ascorbic acid, calcium ascorbate, calcium bisulphite, calcium sulphite, ascorbic acid, isoascorbic acid, potassium metabisulphite, sodium ascorbate, sodium bisulphite, sodium metabisulphite, sodium sulphite, sodium thiosulphate and thioglycerol. The antioxidant synergist may be one or more of citric acid, edetic acid (EDTA) and its salts, hydroxyquinoline sulphate, phosphoric acid, sodium citrate and tartaric acid.

The solution may further include one or more pharmaceutically acceptable additives. The one or more pharmaceutically acceptable additives may be one or more of preservatives, solubilizers, viscosity enhancing agents, colors and flavors. The preservative may be one or more of benzoic acid, sorbic acid, methyl paraben or salts thereof, propyl paraben or salts thereof, benzyl alcohol and benzylalkonium chloride. The buffering agent may be an acid-base combination. The acid may be one or more of succinic, tartaric, lactic, or citric acid and base is sodium hydroxide or disodium hydrogen phosphate. The acid may be tartaric acid and base may be sodium hydroxide.

The flavors may be one or more of vanilla, cherry, raspberry, black currant, strawberry, caramel chocolate, Mint Cool and Fantasy flavors.

In another general aspect there is provided a process for the preparation of an aqueous solution. The process includes mixing water, a therapeutically effective amount of risperidone or a pharmaceutically acceptable free risperidone base or acid addition salt of risperidone, one or more polyhydric alcohols; and one or more buffering agents configured to maintain the pH in the range of about 2 to about 6.

Embodiments of the process may include one or more of the following features. For example, the addition salt may be selected from one or more of salts of risperidone with inorganic acids comprising hydrochloric, hydrobromic, sulfuric, nitric, and phosphoric acids; or organic acids comprising acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, and pamoic acids. The one or more polyhydric alcohols may be one or more of monosaccharides, disaccharides and sugars. The monosaccharide may be one or both of glucose (dextrose) and fructose (levulose). The disaccharide may be one or more of sucrose, lactose, maltose and cellobiose. The disaccharide may be sucrose. The sugars may be one or more of ribose, glycerine, sorbitol, xylitol, maltitol, erythritol, inositol, lactitol monohydrate, propylene glycol, galactose, mannose, xylose, rhamnose, glutaraldehyde, invert sugars, mannitol, polyethylene glycol and glycerol. The sugar may be sorbitol.

The aqueous solution may further include an antioxidant. The antioxidant may be one or more of antioxidants, reducing agents and antioxidant synergist. The antioxidants may be one or more of acetylcysteine, alpha tocopherol acetate, d-alpha tocopherol, dl-alpha tocopherol, ascorbyl palmitate, butylated hydroxyanisole (BHA), butylated hydroxytoluene (BHT), cysteine, cysteine hydrochloride and propyl gallate. The reducing agent may be one or more of ascorbic acid, calcium ascorbate, calcium bisulphite, calcium sulphite, ascorbic acid, isoascorbic acid, potassium metabisulphite, sodium ascorbate, sodium bisulphite, sodium metabisulphite, sodium sulphite, sodium thiosulphate and thioglycerol. The antioxidant synergist may be one or more of citric acid, edetic acid (EDTA) and its salts, hydroxyquinoline sulphate, phosphoric acid, sodium citrate and tartaric acid.

The solution may further include one or more pharmaceutically acceptable additives. The one or more pharmaceutically acceptable additives may be one or more of preservatives, solubilizers, viscosity enhancing agents, colors and flavors. The preservative may be one or more of benzoic acid, sorbic acid, methyl paraben or salts thereof, propyl paraben or salts thereof, benzyl alcohol and benzylalkonium chloride. The buffering agent may be an acid-base combination. The acid may be one or more of succinic, tartaric, lactic, or citric acid and base is sodium hydroxide or disodium hydrogen phosphate. The acid may be tartaric acid and base may be sodium hydroxide.

The flavors may be one or more of vanilla, cherry, raspberry, black currant, strawberry, caramel chocolate, Mint Cool and Fantasy flavors.

In another general aspect, there is provided a method for the management or treatment of the manifestations of psychotic disorders in a mammal. The method includes administering an aqueous solution comprising water; a therapeutically effective amount of risperidone or a pharmaceutically acceptable free risperidone base or acid addition salt of risperidone; one or more polyhydric alcohols; and one or more buffering agents configured to maintain the pH in the range of about 2 to about 6.

Embodiments of the method may include one or more of the following features. For example, the addition salt may be selected from one or more of salts of risperidone with inorganic acids comprising hydrochloric, hydrobromic, sulfuric, nitric, and phosphoric acids; or organic acids comprising acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, and pamoic acids. The one or more polyhydric alcohols may be one or more of monosaccharides, disaccharides and sugars. The monosaccharide may be one or both of glucose (dextrose) and fructose (levulose). The disaccharide may be one or more of sucrose, lactose, maltose and cellobiose. The disaccharide may be sucrose. The sugars may be one or more of ribose, glycerine, sorbitol, xylitol, maltitol, erythritol, inositol, lactitol monohydrate, propylene glycol, galactose, mannose, xylose, rhamnose, glutaraldehyde, invert sugars, mannitol, polyethylene glycol and glycerol. The sugar may be sorbitol.

The aqueous solution may further include an antioxidant. The antioxidant may be one or more of antioxidants, reducing agents and antioxidant synergist. The antioxidants may be one or more of acetylcysteine, alpha tocopherol acetate, d-alpha tocopherol, dl-alpha tocopherol, ascorbyl palmitate, butylated hydroxyanisole (BHA), butylated hydroxytoluene (BHT), cysteine, cysteine hydrochloride and propyl gallate. The reducing agent may be one or more of ascorbic acid, calcium ascorbate, calcium bisulphite, calcium sulphite, ascorbic acid, isoascorbic acid, potassium metabisulphite, sodium ascorbate, sodium bisulphite, sodium metabisulphite, sodium sulphite, sodium thiosulphate and thioglycerol. The antioxidant synergist may be one or more of citric acid, edetic acid (EDTA) and its salts, hydroxyquinoline sulphate, phosphoric acid, sodium citrate and tartaric acid.

The solution may further include one or more pharmaceutically acceptable additives. The one or more pharmaceutically acceptable additives may be one or more of preservatives, solubilizers, viscosity enhancing agents, colors and flavors. The preservative may be one or more of benzoic acid, sorbic acid, methyl paraben or salts thereof, propyl paraben or salts thereof, benzyl alcohol and benzylalkonium chloride. The buffering agent may be an acid-base combination. The acid may be one or more of succinic, tartaric, lactic, or citric acid and base is sodium hydroxide or disodium hydrogen phosphate. The acid may be tartaric acid and base may be sodium hydroxide.

The flavors may be one or more of vanilla, cherry, raspberry, black currant, strawberry, caramel chocolate, Mint Cool and Fantasy flavors.

The details of one or more embodiments of the inventions are set forth in the description below. Other features, objects, and advantages of the inventions will be apparent from the description and the claims.

### DETAILED DESCRIPTION OF THE INVENTION

The inventors have now discovered that stable aqueous solutions of risperidone can be prepared using polyhydric alcohols. U.S. Pat. No. 4,804,663, above, describes an aqueous solution of risperidone with a small amount of water (less than 30% v/v). By experimenting in our laboratory the inventors have discovered that a stable aqueous solution of risperidone containing polyhydric alcohols may be prepared by reducing the solid content and increasing the water concentration of the aqueous solution. The stability may further be improved by incorporating small amounts of an antioxidant. This was confirmed by the accelerated stability data generated at 80° C. over a period of 4 weeks (Table 1). The solution had excellent palatability and could be administered as such, without any further dilution.

The term “stable” as used herein refers to a solution wherein, after storage for a period up to 4 weeks at a temperature of 80° C. or below, the residual amount of risperidone is at least 80% of the initial risperidone concentration.

The term “risperidone” as used herein refers to the free risperidone base as well as pharmaceutically acceptable acid addition salts thereof. Specific examples of acid addition salts include salts with inorganic acids such as hydrochloric acid, hydrobromic acid, sulfuric acid, nitric acid, phosphoric acid and other similar or related inorganic acids; or with organic acids such as acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, pamoic and the like acids. The amount (w/v) of risperidone in the solution may vary from about 0.01% to about 1%, preferably from about 0.02% to about 0.5%, most preferably from about 0.05% to about 0.25%, and in particular is 0.1% (1 mg/l ml).

Examples of polyhydric alcohols that may be used as sweeteners include monosaccharides such as glucose (dextrose) and fructose (levulose); disaccharides such as sucrose, lactose, maltose, and cellobiose; other sugars such as ribose, glycerine, sorbitol, xylitol, maltitol, erythritol, inositol, lactitol monohydrate, propylene glycol, galactose, mannose, xylose, rhamnose, glutaraldehyde, invert sugars, mannitol, polyethylene glycol, glycerol or mixtures thereof. In particular, sucrose and/or sorbitol may be used as sweeteners in an amount (w/v) varying from about 0.01% to about 50%.

Using polyhydric alcohols, such as sorbitol, as a sweetener has many advantages, as these provide bulk and sweetness with a clean, cool pleasant taste. Sorbitol also provides one-third fewer calories than sugar. It is an excellent humectant, texturizing and anti-crystallizing agent. Moreover, polyhydric alcohols are resistant to metabolism by oral bacteria, which break down sugars, and starches that releases acids that may lead to cavities or erode tooth enamel. They are, therefore, non-carcinogenic.

Sorbitol is slowly absorbed, and, consequently, when sorbitol is ingested, the rise in blood glucose and the insulin response which is associated with the ingestion of glucose, is significantly reduced. Therefore Sorbitol can be used as an alternative to sugar for people with diabetes. Sorbitol also has been affirmed as GRAS (Generally Recognized As Safe) by the U.S. Food and Drug Administration and is approved for use by the European Union and numerous countries around the world, including Australia, Canada and Japan.

Sorbitol offers advantages when used in pharmaceutical formulations. For example, sorbitol is very stable, chemically inert and can withstand high temperatures. The commercially available risperidone aqueous solution “Risperdal” has disadvantages, such as the requirement that it be diluted with 100 ml of beverage before consuming. This may be due to the necessity of diluting the bitter taste of risperidone. However, polyhydric alcohols, such as sorbitol, mannitol, fructose, sucrose, and maltose can be used as a bulk sweetener to give a palatable aqueous solution that can be administered without any dilution. Consequently, the use of polyhydric alcohols, particularly sorbitol, which are highly effective as sweeteners help in better taste masking of the bitter taste of risperidone and thereby do not necessitate dilution during administration

Antioxidants used for enhancing the stability of risperidone solution include compounds from any of the three general classes of antioxidant: true antioxidants, reducing agents, and antioxidant synergist. Examples of suitable true antioxidants include acetylcysteine, alpha tocopherol acetate, d-alpha tocopherol, dl-alpha tocopherol, ascorbyl palmitate, butylated hydroxyanisole (BHA), butylated hydroxytoluene (BHT), cysteine, cysteine hydrochloride, propyl gallate and the like. Examples of suitable reducing agents include ascorbic acid, calcium ascorbate, calcium bisulphite, calcium sulphite, isoascorbic acid, potassium metabisulphite, sodium ascorbate, sodium bisulphite, sodium metabisulphite, sodium sulphite, sodium thiosulphate, thioglycerol and the like. Examples of suitable antioxidant synergists include citric acid and edetic acid (EDTA) and its salts, including disodium EDTA, hydroxyquinoline sulphate, phosphoric acid, sodium citrate, tartaric acid and the like.

In particular, antioxidants are used that are safe for oral ingestion and have sufficient solubility in the solution to make a stable, single-phase composition which is stable over a wide range of temperatures and pH values and is compatible with other components of the solution. Mixtures of two or more of the antioxidants may also be used. The amount (w/v) of antioxidant may vary from about 0.01% to about 5.0%.

Besides the above, the stable aqueous solution of risperidone may also include one or more pharmaceutically acceptable additives such as antimicrobial preservatives, buffering agents, solubilizers, viscosity enhancing agents, colors, flavors and the like.

Examples of suitable preservatives include benzoic acid, sorbic acid, and methyl paraben or salts thereof, propyl paraben or salts thereof, benzyl alcohol and benzylalkonium chloride. The concentration (w/v) of the preservative may range from about 0.05% to about 2%.

Examples of suitable solubilizers include co-solvents, complexing agents, surfactants wetting agents and the like.

Examples of suitable viscosity enhancing agents include hydroxypropyl methylcellulose (some forms of which are available from Dow Chemical, Midland, Mich. USA under the METHOCEL trademark), hydroxpropyl cellulose and the like.

Examples of suitable colors and flavors include all FDA approved colors or flavors suitable for oral use. Specific examples of flavors include vanilla, cherry, raspberry, black currant, strawberry, Caramel Chocolate, Mint Cool, Fantasy flavors and the like.

If desired, the pH of the stable risperidone solution may be adjusted from between about 2 to about 6, with the use of buffering agents. Buffering agents are acid-base combinations such as succinic, tartaric, lactic, or citric acid with sodium hydroxide or disodium hydrogen phosphate.

In one of the embodiments a stable aqueous risperidone solution may be prepared by:
(a) dissolving preservatives, stabilizers and acid component of the buffering system in hot purified water;
(b) cooling the solution;
(c) dissolving risperidone under continuous stirring in the cooled solution;
(d) adding one or more sweeteners;
(e) adding one or more colors and/or flavors; and
(f) adjusting the pH with the basic component of the buffering system and making up the volume.

The following examples are intended to illustrate the scope of the present invention in all its aspects but not to limit it thereto.

Example 1

| Ingredients | Quantity | |
|---|---|---|
| Risperidone | 1 | mg/ml |
| Benzoic Acid | 1 | mg/ml |
| Tartaric Acid | 7.5 | mg/ml |
| Sodium hydroxide | 2 | mg/ml |
| Sorbitol Solution (70%) | 10% | w/v |
| Artificial Creme De Vanilla Flavor | 0.15% | v/v |
| Artificial Raspberry Flavor | 0.5% | v/v |

| | |
|---|---|
| Purified water | q.s to 1 ml |

Process:
1. Benzoic acid was dissolved in purified water at 60° C.
2. Tartaric acid was dissolved in the solution of step 1, and then cooled to a temperature of less than 30° C.
3. Risperidone was then dissolved in the cooled solution under continuous stirring.
4. Sorbitol solution (70%) was mixed with the bulk solution of step 3, followed by the addition of Artificial Creme De Vanilla Flavor and Artificial Raspberry Flavor.
5. The pH of the solution of step 4 was then adjusted to between about 3 and about 4 with sodium hydroxide solution, followed by volume makeup using purified water.

6. The bulk of step 5 was then filtered through a 5 μm polypropylene filter and filled into suitable containers.

Example 2

| Ingredients | Quantity | |
|---|---|---|
| Risperidone | 1 | mg/ml |
| Benzoic Acid | 1 | mg/ml |
| Tartaric Acid | 7.5 | mg/ml |
| Sodium hydroxide | 2 | mg/ml |
| Soibitol Solution (70%) | 10% | w/v |
| EDTA disodium | 1 | mg/ml |
| Artificial Creme De Vanilla Flavor | 0.15% | v/v |
| Artificial Raspberry Flavor | 0.5% | v/v |

| | |
|---|---|
| Purified water | q.s to 1 ml |

Process:
1. Benzoic acid and EDTA disodium were dissolved in purified water at 60° C.
2. Tartaric acid was dissolved in the solution of step 1, and then cooled to a temperature of less than 30° C.
3. Risperidone was then dissolved in the cooled solution under continuous stirring.
4. Sorbitol solution (70%) was mixed with the bulk solution of step 3, followed by the addition of Artificial Creme De Vanilla Flavor and Artificial Raspberry Flavor.
5. The pH of the solution of step 4 was then adjusted to between about 3 and about 4 with sodium hydroxide solution, followed by volume makeup using purified water.
6. The bulk of step 5 was then filtered through a 5 μm polypropylene filter and filled into suitable containers.

The above solutions when subjected to accelerated stability, i.e., at a temperature of 80° C. for a period up to 4 weeks, showed excellent stability. This is clearly evident from the data given in Table-1.

TABLE 1
Accelerated stability data of risperidone solutions containing
sorbitol generated at 80° C. for a period of four weeks

Risperidone concentration (%)

| Formulation | Initial | After 4 weeks at 80° C. |
|---|---|---|
| Solution with 70% sorbitol | 100 | <80 |
| Solution with 10% sorbitol | 99.90 | 88.60 |
| (Example 1) | | |
| Solution with 10% sorbitol | 102.50 | 96.20 |
| and EDTA di-sodium | | |
| (Example 2) | | |

While several particular forms of the inventions have been described, it will be apparent that various modifications and combinations of the inventions detailed in the text can be made without departing from the spirit and scope of the inventions. For example, although the examples above are directed to application of the inventive concepts described herein to risperidone as the active pharmaceutical ingredient, these concepts can be applied to other active antipsychotic ingredients, such as 1,2-benzisoxazol-3-yl derivatives. Finally, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. An aqueous solution of risperidone, the aqueous solution comprising:
water;
a therapeutically effective amount of risperidone or a pharmaceutically acceptable free risperidone base or acid addition salt of risperidone;
one or more polyhydric alcohols; and
one or more buffering agents configured to maintain the pH in the range of about 3 to about 4.

2. The aqueous solution of claim 1, wherein the addition salt is selected from one or more of salts of risperidone with inorganic acids comprising hydrochloric, hydrobromic, sulfuric, nitric, and phosphoric acids; or organic acids comprising acetic, propanoic, hydroxyacetic, lactic, pyruvic, oxalic, malonic, succinic, maleic, fumaric, malic, tartaric, citric, methane-sulfonic, ethanesulfonic, benzenesulfonic, p-toluenesulfonic, cyclamic, salicylic, p-aminosalicylic, and pamoic acids.

3. The aqueous solution of claim 1 wherein the one or more polyhydric alcohols comprises one or more of monosaccharides, disaccharides and sugars.

4. The aqueous solution of claim 3 wherein the monosaccharide comprises one or both of glucose (dextrose) and fructose (levulose).

5. The aqueous solution of claim 3 wherein the disaccharide comprises one or more of sucrose, lactose, maltose and cellobiose.

6. The aqueous solution of claim 3 wherein the disaccharide comprises sucrose.

7. The aqueous solution of claim 3 wherein the sugars comprise one or more of ribose, glycerine, sorbitol, xylitol, maltitol, erythritol, inositol, lactitol monohydrate, propylene glycol, galactose, mannose, xylose, rhamnose, glutaraldehyde, invert sugars, mannitol, polyethylene glycol and glycerol.

8. The aqueous solution of claim 3 wherein the sugar comprises sorbitol.

9. The aqueous solution of claim 1 wherein the aqueous solution further comprises an antioxidant.

10. The aqueous solution of claim 9 wherein the antioxidant comprises one or more of antioxidants, reducing agents and antioxidant synergist.

11. The aqueous solution of claim 10 wherein the antioxidants comprises one or more of acetylcysteine, alpha tocopherol acetate, d-alpha tocopherol, dl-alpha tocopherol, ascorbyl palmitate, butylated hydroxyanisole (BHA), butylated hydroxytoluene (BHT), cysteine, cysteine hydrochloride and propyl gallate.

12. The aqueous solution of claim 10 wherein the reducing agent comprises one or more of ascorbic acid, calcium ascorbate, calcium bisulphite, calcium sulphite, ascorbic acid, isoascorbic acid, potassium metabisulphite, sodium ascorbate, sodium bisulphite, sodium metabisulphite, sodium sulphite, sodium thiosulphate and thioglycerol.

13. The aqueous solution of claim 10 wherein the antioxidant synergist comprises one or more of citric acid, edetic acid (EDTA) and its salts, hydroxyquinoline sulphate, phosphoric acid, sodium citrate and tartaric acid.

14. The aqueous solution of claim 1 wherein the solution further comprises one or more pharmaceutically acceptable additives.

15. The aqueous solution of claim 14 wherein the one or more pharmaceutically acceptable additives comprise one or more of preservatives, solubilizers, viscosity enhancing agents, colors and flavors.

16. The aqueous solution of claim 15 wherein the preservative comprises one or more of benzoic acid, sorbic acid, methyl paraben or salts thereof, propyl paraben or salts thereof, benzyl alcohol and benzylalkonium chloride.

17. The aqueous solution of claim 1 wherein the buffering agent comprises an acid-base combination.

18. The aqueous solution of claim 17 wherein the acid comprises one or more of succinic, tartaric, lactic, or citric acid and base is sodium hydroxide or disodium hydrogen phosphate.

19. The aqueous solution of claim 17 wherein the acid is tartaric acid and base is sodium hydroxide.

20. The aqueous solution of claim 15 wherein the flavors comprise one or more of vanilla, cherry, raspberry, black currant, strawberry, caramel chocolate, Mint Cool and Fantasy flavors.

21. A process for the preparation of an aqueous solution, the process comprising:
mixing water, a therapeutically effective amount of risperidone or a pharmaceutically acceptable free risperidone base or acid addition salt of risperidone, one or more polyhydric alcohols; and one or more buffering agents configured to maintain the pH in the range of about 3 to about 4.

22. A method for the management or treatment of the manifestations of psychotic disorders in a mammal, the method comprising administering an aqueous solution comprising water; a therapeutically effective amount of risperidone or a pharmaceutically acceptable free risperidone base or acid addition salt of risperidone; one or more polyhydric alcohols; and one or more buffering agents configured to maintain the pH in the range of about 3 to about 4.
